(19) United States
(12) Patent Application Publication
(10) Pub. No.: US 2025/0325833 A1
(43) Pub. Date: Oct. 23, 2025

(54) ANATOMICAL SEPARATOR FOR MEDICAL TREATMENTS

(71) Applicants: Thomas Furno, Bradenton, FL (US); Ramtin Zakikhani, Sarasota, FL (US); Eric Arjen Winkel, Almere (NL)

(72) Inventors: Thomas Furno, Bradenton, FL (US); Ramtin Zakikhani, Sarasota, FL (US); Eric Arjen Winkel, Almere (NL)

(21) Appl. No.: 19/078,965

(22) Filed: Mar. 13, 2025

Related U.S. Application Data
(60) Provisional application No. 63/636,721, filed on Apr. 20, 2024.

(51) Int. Cl. A61N 5/10

(52) U.S. Cl. CPC A61N 5/10; A61N 2005/1092

(57) ABSTRACT

A separator for medical applications includes a first contact surface configured for contacting a first anatomical surface of a patient to be imaged, and a second contact surface configured for contacting a second anatomical surface of the patient to be imaged to space the first anatomical surface from the second anatomical surface. The first contact surface is angled relative to the second contact surface. The separator maintains spacing between anatomical surfaces during radiotherapy procedures, potentially reducing radiation exposure to sensitive areas. The separator may comprise air equivalent or radiotransparent materials and can be used in various anatomical regions where skin folds or adjacent body parts need separation for improved therapy results.

### CROSS-REFERENCE TO RELATED APPLICATIONS

This application claims priority to U.S. Provisional Application No. 63/636,721, titled “Inframammary Fold Breast Wedge,” filed Apr. 20, 2024, which is hereby incorporated by reference in its entirety.

### FIELD OF INVENTION

The present disclosure relates to medical treatment devices and systems, and more particularly to an anatomical separator for creating space between body regions during radiotherapy and/or other medical procedures.

### BACKGROUND

Radiotherapy is a widely used treatment modality for various types of cancer, including breast cancer. During radiotherapy treatments, precise delivery of radiation to the target area while minimizing exposure to surrounding healthy tissues is crucial for optimal outcomes. However, the natural anatomy of the human body often presents challenges in achieving this goal.

In many radiotherapy setups, patients are positioned in a supine or prone position on a treatment table. This positioning can result in skin folds or areas where different anatomical surfaces come into close contact or overlap. For example, in breast cancer radiotherapy, the breast tissue may lie in direct contact with the chest wall, creating an inframammary fold. Similar situations can occur in other body regions, such as the axilla or groin area.

These areas of skin-to-skin contact or overlapping tissues can pose several problems during radiotherapy. For instance, these overlapping tissues may receive higher doses of radiation due to the lack of separation between surfaces, potentially leading to increased skin toxicity, discomfort, and/or undesired cosmetic outcomes. Additionally, the lack of space between anatomical surfaces can make it challenging to accurately target the intended treatment area while sparing nearby healthy tissues.

Existing approaches to address these issues have included various positioning aids and immobilization wherein the separator is configured to maintain spacing between the first anatomical surface and the second anatomical surface during treatment. However, these conventional solutions are not specifically designed to create separation between adjacent anatomical surfaces in a way that is both effective and comfortable for the patient. Furthermore, some positioning devices may introduce additional complexities to the treatment setup or treatment process.

Accordingly, there is a need for improved methods and devices to create and maintain space between anatomical surfaces during medical procedures such as radiotherapy procedures. There also is a need for solutions that are simple to use, adaptable to different body regions, and/or compatible with existing medical equipment. There also is a need for solutions designed to minimize interference with radiation beams while providing consistent and reproducible patient positioning across multiple treatment sessions.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a separator for medical procedures is provided. The separator includes a first contact surface configured for contacting a first anatomical surface of a patient to be treated. The separator also includes a second contact surface configured for contacting a second anatomical surface of the patient to be treated to space the first anatomical surface from the second anatomical surface. The first contact surface is angled relative to the second contact surface.

According to other aspects of the present disclosure, the separator may include one or more of the following features. The first contact surface may extend from a first arcuate surface to a second arcuate surface, and the second contact surface may extend from the first arcuate surface to the second arcuate surface. The first arcuate surface may be substantially parallel to the second arcuate surface. At least one of the first contact surface or the second contact surface may extend along an arc between a first end wall and a second end wall. The arc may be between about 90-degrees and about 190-degrees. The first contact surface may be angled relative to the second contact surface by an angle of from about 2-degrees to about 30-degrees. The separator may comprise at least one of an air equivalent material or a radiotransparent material. The at least one of the air equivalent material or the radiotransparent material may comprise at least one of foam, polyurethane foam, or a polymeric material.

According to additional aspects of the present disclosure, methods of treating a patient are provided. These methods may include providing a separator comprising a first contact surface and a second contact surface angled relative to the first contact surface. The method may also include placing the separator proximate a patient such that the first contact surface contacts a first anatomical surface of the patient and the second contact surface contacts a second anatomical surface of the patient. Thus positioned, the anatomical separator retains the first anatomical surface spaced from the second anatomical surface. The methods described herein may further include treating the patient by applying radiation proximate the first anatomical surface, wherein the separator reduces a dose of the radiation at the first anatomical surface and the second anatomical surface.

According to another aspect of the present disclosure, a system for medical treatment is provided. The system includes a medical apparatus configured to emit radiation and a patient support. The system also includes a separator positioned on the patient support. The separator comprises a first contact surface configured to contact a first anatomical surface of a patient and a second contact surface angled relative to the first contact surface and configured to contact a second anatomical surface of the patient. The separator is configured to maintain spacing between the first anatomical surface and the second anatomical surface during radiation of the patient.

The foregoing general description of the illustrative examples of this disclosure and the following detailed description thereof are merely exemplary aspects of the teachings of this disclosure and are not restrictive.

### BRIEF DESCRIPTION OF FIGURES

The detailed description is described with reference to the accompanying figures. In the figures, the left-most digit(s) of a reference number identifies the figure in which the reference number first appears. The use of the same reference numbers in different figures indicates similar or identical components or features.

FIG. 1 illustrates a perspective view of a treatment scenario in which an example anatomical separator is used, according to aspects of the present disclosure.

FIG. 2A illustrates a perspective view of an anatomical separator for medical applications, according to aspects of this disclosure.

FIG. 2B illustrates a top orthogonal view of the separator of FIG. 2A, according to aspects of the present disclosure.

FIG. 2C illustrates a cross-sectional side view of the separator of FIG. 2A in use with a patient, according to aspects of this disclosure.

FIG. 3A illustrates a perspective view of an anatomical separator for medical applications, according to aspects of the present disclosure.

FIG. 3B illustrates a top orthogonal view of the separator of FIG. 3A, according to aspects of this disclosure.

FIG. 3C illustrates a cross-sectional side view of the separator of FIG. 3A in use with a patient, according to aspects of the present disclosure.

FIG. 4 illustrates an orthogonal cross-sectional side view of another example anatomical separator, according to an aspect of the present disclosure.

FIG. 5 illustrates an orthogonal cross-sectional side view of another example anatomical separator, according to aspects of the present disclosure.

FIG. 6 illustrates a perspective view of an anatomical separator device for medical applications in use with a patient, according to aspects of the present disclosure.

FIG. 7 is a flowchart showing operations associated with a process for treating a patient using an anatomical separator, according to aspects of this disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

As discussed above, the present disclosure relates to anatomical separators for use in medical procedures such as radiotherapy procedures. In examples of this disclosure, anatomical separators may be used to create and maintain space between different body regions during treatment, e.g., during radiation treatment. In some aspects of this disclosure, an anatomical separator includes a first contact surface and a second contact surface that are angled relative to each other. The first contact surface may be configured to contact a first anatomical surface of a patient, while the second contact surface may be configured to contact a second anatomical surface of the patient.

In certain implementations, the angled configuration of the contact surfaces allows the anatomical separator to create separation between adjacent body regions that may otherwise be in close contact. This separation may be beneficial in various medical treatment scenarios. For example, during radiotherapy for breast cancer, an anatomical separator may be positioned to create space between the breast tissue and the chest wall.

Anatomical separators according to the present disclosure may be constructed from materials that are compatible with medical procedures, including but not limited to radiotherapy and/or other treatment procedures. In some cases, the separators may comprise air equivalent or radiotransparent materials. Such materials may allow radiation beams to pass through the separator with minimal interference or attenuation.

The shape and dimensions of the anatomical separators may be varied to suit different anatomical regions and patient needs. In some implementations, the contact surfaces may extend between arcuate surfaces. The angular relationship between the contact surfaces may also be adjusted to provide optimal spacing for particular applications.

Anatomical separators as described herein may offer several potential benefits in medical treatment and radiotherapy contexts. By creating space between adjacent anatomical surfaces, the separators may help reduce radiation dosages to skin folds and other areas where tissues may otherwise be in direct contact. Additionally, the separators may assist in positioning patients and maintaining consistent setup across multiple treatment sessions.

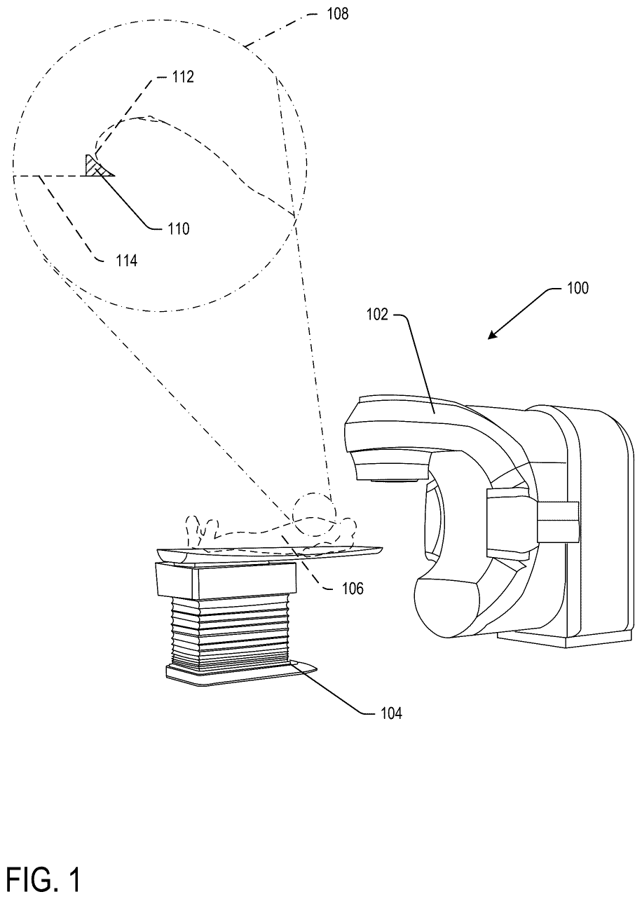

Referring to FIG. 1, a treatment scenario 100 for medical treatment is illustrated. The treatment scenario 100 may include a radiation emitting apparatus 102 configured to emit radiation for treatment purposes. In some examples, the radiation emitting apparatus 102 may be a radiotherapy system, a computed tomography (CT) scanner, or a type of medical treatment device capable of emitting radiation for diagnostic or therapeutic purposes.

The treatment scenario 100 may also include a patient support 104. In some cases, the patient support 104 may be a table or platform designed to position and support a patient 106 during treatment procedures. The patient support 104 may be adjustable to facilitate proper positioning of the patient 106 relative to the treatment apparatus 102.

In the illustrated example, the patient 106 may be positioned in a supine position on the patient support 104. The radiation emitting apparatus 102 may be configured to irradiate the patient 106 from above. This arrangement may allow for targeted radiation delivery to specific anatomical regions while the patient 106 remains in a comfortable and stable position.

The supine position, while commonly used in medical imaging and treatment procedures, may present certain challenges. In some cases, when a patient is positioned supine on a flat surface, various anatomical regions may come into close contact or overlap. This skin-to-skin contact may occur in areas such as the inframammary fold, axilla, or between adjacent skin surfaces of the abdomen or thighs.

The close proximity or direct contact between skin surfaces in the supine position may lead to several issues during medical procedures. In some instances, these areas of skin contact may trap moisture, potentially increasing the risk of skin irritation or breakdown over time. Additionally, in radiotherapy contexts, regions of skin-to-skin contact may receive higher doses of radiation due to the lack of air gap between surfaces, which may increase the likelihood of skin toxicity or other adverse effects. Furthermore, the supine position may cause certain anatomical structures to compress or deform under the influence of gravity. For example, breast tissue may flatten against the chest wall, potentially altering the target volume for radiation treatment. This compression may also create challenges in accurately delineating treatment boundaries and ensuring consistent dose delivery to the intended areas. In some cases, the supine position may also or additionally result in the formation of skin folds, particularly in patients with higher body mass indices. These skin folds may create regions of variable tissue thickness, which may complicate dose calculations and delivery in radiotherapy applications. The presence of skin folds may also increase the risk of skin toxicity in these areas due to potential dose build-up effects.

FIG. 1 also includes a magnified portion 108 showing that, in examples of this disclosure, a separator 110 is used with the patient 106 to mitigate some or all of the problems associated with treating the patient 106 in the supine position. Specifically, the magnified portion 108 shows that the separator 110 may be placed between two anatomical surfaces of the patient 106. For example, the separator 110 may be positioned between a first anatomical surface 112 and a second anatomical surface 114 of the patient 106. In the illustrated example, the first anatomical surface 112 may be a surface of a breast of the patient 106 and the second anatomical surface 114 may be a surface of a chest or abdomen of the patient 106. In this example, the surfaces 112, 114 create an inframammary fold. As illustrated in the magnified portion 108. the separator 110 may be positioned within this inframammary fold to maintain separation between the surfaces 112 and 114, potentially reducing skin-to-skin contact in this area during treatment.

Separating the anatomical surfaces 112, 114 with the separator 110 may serve multiple purposes in the treatment scenario 100. In some cases, the separator 110 may help reduce the dose of radiation at the first anatomical surface 112 and/or the second anatomical surface 114. This reduction in radiation dose may be achieved by creating physical separation between the anatomical surfaces, potentially reducing scatter radiation or direct exposure to certain areas.

The positioning of the separator 110 may vary depending on the specific medical procedure and anatomical region of interest. For instance, in the example of FIG. 1 the separator 110 may be placed in the inframammary fold region during breast treatment. In other examples, however, the separator 110 may be used in other anatomical regions where tissue separation is desired for improved outcomes, including reduced radiation exposure.

The treatment scenario 100 may be configured to allow for various procedures while utilizing the separator 110. In some cases, the radiation emitting apparatus 102 may emit radiation that passes through or near the separator 110 to capture images of the patient 106. The separator 110 may be composed of materials that are compatible with the treatment modality, such as radiotransparent or air-equivalent materials, to minimize interference with the treatment process.

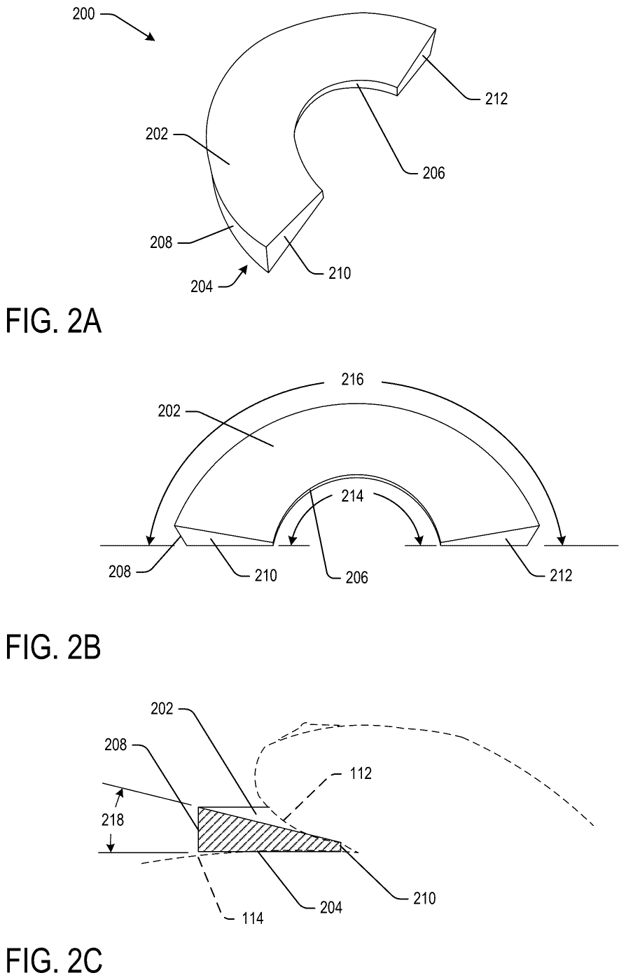

Referring to FIGS. 2A-2C, various views of a separator 200 for medical treatment are illustrated. The separator 200 may be an example of the separator 110 just described. The separator 200 may include a first contact surface 202 and an opposing second contact surface 204. In use, the first contact surface 202 is configured for contacting a first anatomical surface, like the first anatomical surface 112, and the second contact surface 204 is configured for contacting a second anatomical surface, like the second anatomical surface 114. In examples, the first contact surface 202 may be an upper surface e.g., configured for contacting a patient's breast when the patient is positioned in a supine position, and the second contact surface 204 may be a lower surface, e.g., configured for contacting a patient's abdomen or chest, generally as shown in FIG. 2C.

In some examples, the separator is generally C- or U-shaped, such that the first contact surface 202 extends from a first arcuate surface 206 to a second arcuate surface 208. Similarly, the second contact surface 204 may extend from the first arcuate surface 206 to the second arcuate surface 208. In some examples, the arcuate surfaces 206, 208 may be formed at some radius from an imaginary point or axis. Moreover, in certain implementations, the first arcuate surface 206 may be substantially parallel to the second arcuate surface 208. In other examples, however, the first arcuate surface 206 and the second arcuate surface 210 may be other than parallel. When used in the application illustrated in FIG. 2C, e.g., in the inframammary fold, the first arcuate surface 206 may be contoured generally to conform to the shape of a breast. In this example, the second arcuate surface 208 may take any shape, including non-arcuate shapes, inasmuch as the second arcuate surface 208 may be uninvolved in the desired function of separating the first and second anatomical surfaces 112, 114. That is, in addition to not being parallel to the first arcuate surface 206, the second arcuate surface 208 may be other than arcuate.

As best illustrated in FIGS. 2A and 2B, the separator 200 also includes a first end wall 210 and a second end wall 212. In the illustrated examples, the first contact surface 202, the second contact surface 204, the first arcuate surface 208 and the second arcuate surface 210 extend along an arc between the first end wall 210 and the second end wall 212. FIG. 2B shows that the first arcuate surface 208 has a first arcuate surface angular extent 214 and the second arcuate surface 210 has a second arcuate surface angular extent 216. These angular extents are generally illustrated as angles about an imaginary point or axis about which the associated arcuate surfaces 208, 210 may be disposed. The angular extents 214, 216 may vary depending on the specific application and anatomical region for which the separator 200 is designed. In certain aspects, the arcs may be between about 45-degrees and about 190-degrees. Moreover, although the example of FIG. 2B generally shows that the first arcuate surface angular extent 214 and the second arcuate surface angular extent 216 are generally equal, e.g., with each of the first end wall 210 and the second end wall 212 extending generally radially from the imaginary point or axis discussed above, in other examples the end walls 210. 212 may be formed at an angle relative to such a radius.

As also illustrated in FIGS. 2A-2C, the first contact surface 202 and the second contact surface 204 may be angled relative to each other, e.g., by an angular displacement 218. This angled configuration may allow the separator 200 to create and maintain space between different anatomical surfaces during medical procedures. The specific angle between the contact surfaces may be selected based on the desired degree of separation and the particular anatomical region where the separator 200 will be used. In some examples, the angular displacement 218 may be between about 2 degrees and about 40-degrees, although smaller or larger angular displacements may also be used.

In some implementations, the curved geometry of the separator 200 may allow it to conform to various anatomical contours while still maintaining the desired spacing between surfaces. The combination of curved surfaces and angled contact surfaces may provide flexibility in positioning and use across different patient anatomies and medicinal treatment scenarios.

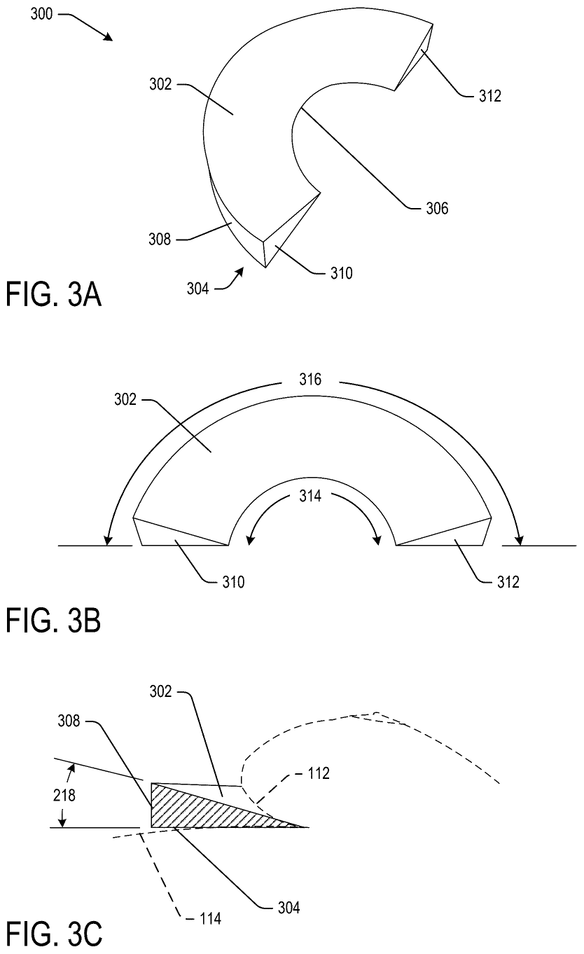

Referring to FIGS. 3A-3C, various views of an alternative separator 300 for medical treatments are illustrated. Like the separator 200, the separator 300 may be an example of the separator 110, discussed above. The separator 300 may include a first contact surface 302 and a second contact surface 304. In some examples, the first contact surface 302 may extend from a first arcuate edge 306 to a second arcuate surface 308. The second contact surface 304 may also extend between the first arcuate edge 306 and the second arcuate surface 308. Thus, the separator 300 may be similar to the separator 200, but may have a first arcuate edge 306 instead of the first arcuate surface 206. This modification will reduce a thickness of the separator 300 proximate the junction of the first contact surface 302 and the second contact surface 304.

Like the separator 200, the separator 300 may also include a first end wall 310 and a second end wall 312. In some cases, at least one of the first contact surface 302 or the second contact surface 304 may extend along an arc between the first end wall 310 and the second end wall 312. The angular extent of this arc may vary depending on the specific application and anatomical region for which the separator 300 is designed. In certain aspects, the arc may be between about 45-degrees and about 190-degrees, as indicated by the first arcuate edge angular extent 314 and the second arcuate surface angular extent 316.

The first contact surface 302 and the second contact surface 304 may be angled relative to each other, creating an angular displacement 318. This angled configuration may allow the separator 300 to create and maintain space between different anatomical surfaces during treatment procedures. The specific angle between the contact surfaces may be selected based on the desired degree of separation and the particular anatomical region where the separator 300 may be used. FIG. 3A shows an application of the separator 300 in an inframammary fold, as discussed herein.

In some implementations, the separator 300 may differ from the separator 200 shown in FIGS. 2A-2C in terms of its overall geometry and the relationship between its surfaces. For example, the first arcuate edge 306 of separator 300 may have a smaller radius of curvature compared to the first arcuate surface 206 of separator 200. This difference in curvature may allow the separator 300 to conform to different anatomical contours or to be used in different anatomical regions.

The second arcuate surface 308 of the separator 300 may extend further laterally compared to the second arcuate surface 208 of separator 200. This extended surface may provide additional support or contact area in certain applications. The relationship between the first contact surface 302 and the second contact surface 304 may also differ from that of separator 200, potentially allowing for different spacing configurations between anatomical surfaces.

In some examples, the separator 300 may be designed with a more pronounced wedge-like profile when viewed from the side, as compared to separator 200. This profile may be advantageous for creating graduated spacing between anatomical surfaces in certain treatment scenarios. The specific shape and dimensions of the separator 300 may be tailored to suit particular treatment needs and/or patient anatomies.

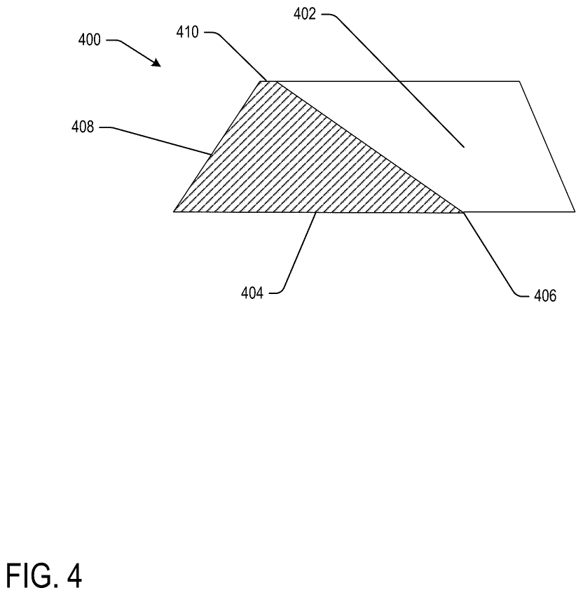

Referring to FIG. 4, a separator 400 for medical treatment is illustrated. Like the separators 200, 300 described above, the separator 400 may be an example of the separator 110. The separator 400 may include a first contact surface 402 for contacting a first anatomical surface and a second contact surface 404 for contacting a second anatomical surface. In some examples, the first contact surface 402 may be angled relative to the second contact surface 404. The angle between the first contact surface 402 and the second contact surface 404 may vary depending on the specific application and anatomical region where the separator 400 is to be used. In certain implementations, this angle may be from about 2-degrees to about 30-degrees.

The separator 400 may have a wedge-shaped design, with the first contact surface 402 and the second contact surface 404 converging towards one edge. This configuration may allow for graduated spacing between anatomical surfaces when the separator 400 is in use. In some cases, the first contact surface 402 may be substantially flat, while the second contact surface 404 may be angled or curved.

The separator 400 may also include a first arcuate edge 406 and a second arcuate surface 408, like the example shown in FIGS. 3A-3C. These curved elements may allow the separator 400 to conform to various anatomical contours while maintaining the desired spacing between surfaces. In some implementations, the separator 400 may include a lip 410 at one end, which may assist in positioning or securing the separator 400 during use. in examples, the second arcuate surface 408 may be angled relative to the first contact surface 402 and the second contact surface 404. For instance, the second arcuate surface 408 and the first contact surface 402 may be formed to create a desired profile relative to the second contact surface 404. For instance, while outer arcuate surfaces 206, 306 described above are generally perpendicular to the respective second contact surfaces 204, 304, the second arcuate surface 408 of the separator 400 is disposed at an acute angle relative to the second contact surface 404.

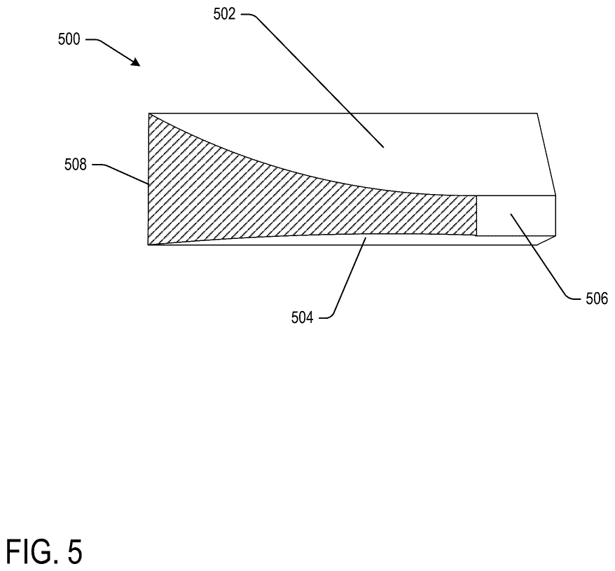

Referring to FIG. 5, a cross-sectional view of another anatomical separator 500 for use in medical procedures is illustrated. Like other separators described herein, the separator 500 may include a first contact surface 502 and a second contact surface 504 spaced from the first contact surface 502. As also illustrated in FIG. 5, the contact surfaces 502, 504 extend between a first (inner) arcuate surface 506 and a second (outer) arcuate surface 508. Unlike other examples in which the first and second contact surfaces are generally planar, in the example separator 500, the first contact surface 502 and the second contact surface 504 are concave.

Like in other examples, the wedge-shaped design of the separator 500 may allow for creating a graduated space between two anatomical surfaces during medical treatment procedures. In some cases, the flat surface may contact one anatomical surface while the angled surface contacts another, maintaining separation between them. The angle between the first contact surface and the second contact surface may vary depending on the specific application and desired degree of separation. In some examples, these concave surfaces may better conform to anatomical structures than planar surfaces.

According to the foregoing, aspects of this disclosure relate to a number of different anatomical separators, having different shapes, sizes, and configurations. For example, one of the example separators described herein may be selected based on the medical application, a region of the patient to be treated, an anatomy of the patient, and/or other features.

The material composition of the separators may be selected to optimize their performance in medical applications. In some examples, the separator 400 may comprise at least one of an air equivalent material or a radiotransparent material. These materials may allow radiation to pass through the separator with minimal interference or attenuation during treatment procedures. In certain implementations, the air equivalent material or radiotransparent material may include at least one of foam, polyurethane foam, or a polymeric material. For example, the separators described herein may be constructed from medical-grade polyurethane foam. The use of such materials may provide the necessary structural support while minimizing the impact on treatment quality.

In some examples, the separator may be constructed from a variety of materials beyond foam or polyurethane. For example, the separator may be made of plastic, high-density polyethylene (HDPE), bubble wrap, rubber, or balloon material. The choice of material may depend on factors such as the specific treatment modality, patient comfort, and whether the separator is intended for single-use or multiple uses.

The shape of the separator may vary to accommodate different anatomical regions and patient needs. In some cases, the separator may have a straight design, while in other implementations, the separator may have a circular, half-circle, or U-shaped configuration. These different shapes may allow the separator to conform to various anatomical contours while still maintaining the desired spacing between surfaces.

The dimensions of the separator may also be customized for specific applications. In some examples, the separator may have a length at the cross-section ranging from about 1 cm to about 15 cm. This range of lengths may allow for separators suitable for use in various anatomical regions, from smaller areas like the axilla to larger regions such as the breast or abdomen. The height of the separator may vary as well, potentially ranging from about 1 cm to about 5 cm. This range of heights may provide options for creating different degrees of separation between anatomical surfaces.

In some implementations, the separator may be designed disposable, e.g., designed for a single use. In other examples, however, the separators described herein may be reusable. To facilitate this, the separator may be made from and/or coated with a material that allows for easy cleaning and sanitization between uses. The specific coating may be selected based on factors such as compatibility with cleaning agents, durability, and potential impact on treatment quality. The ability to sanitize and reuse the separator may offer cost-effective and environmentally friendly options for healthcare providers.

In some implementations, the separator may be coated with medically safe and sterile materials to enhance its suitability for reuse in clinical settings. For example, the separator may be coated with a medical-grade silicone material, which may provide a smooth, non-porous surface that is easy to clean and sterilize. Silicone coatings may also offer good biocompatibility and resistance to degradation from repeated cleaning and sterilization processes. Another option for coating the separator may be a polyurethane-based material. Polyurethane coatings may provide durability and flexibility while maintaining compatibility with various sterilization methods. These coatings may also offer good chemical resistance, potentially extending the usable life of the separator.

In some cases, the separator may be coated with a hydrophobic material such as fluoropolymers. These coatings may help repel liquids and reduce the adherence of contaminants, potentially simplifying the cleaning process. Fluoropolymer coatings may also provide resistance to chemicals commonly used in medical environments. Antimicrobial coatings may also be applied to the separator in some implementations. For instance, coatings containing silver nanoparticles or quaternary ammonium compounds may help reduce the risk of microbial growth on the separator's surface between uses. These antimicrobial properties may complement standard cleaning and sterilization procedures.

In other aspects, the separator may be coated with a medical-grade epoxy resin. Epoxy coatings may provide a hard, durable surface that is resistant to scratches and chemical damage. This durability may be beneficial for separators that undergo frequent handling and reprocessing. Some implementations may utilize a parylene coating on the separator. Parylene coatings may offer excellent barrier properties and biocompatibility. The thin, conformal nature of parylene coatings may allow for precise application without significantly altering the dimensions or properties of the underlying separator material. In certain cases, the separator may be coated with a medical-grade acrylic material. Acrylic coatings may provide good optical clarity, which may be advantageous in scenarios where visual inspection of the separator is important. These coatings may also offer good resistance to yellowing over time, potentially maintaining the aesthetic appearance of the separator through multiple use cycles.

The combination of material options, shape variations, and dimensional flexibility may allow for a wide range of separator designs tailored to specific medical treatment and radiotherapy applications. These design variations may enable healthcare providers to select or customize separators that best meet the needs of individual patients and particular treatment scenarios.

As shown in the examples of FIGS. 1, 2C, and 3C, aspects of this disclosure may be particularly well suited for use in breast treatments, e.g., by being positioned to provide spacing in an inframammary fold. However, this disclosure is not limited to use in any one application. As described herein, many medical treatments may benefit from reduction of skin-on-skin contact.

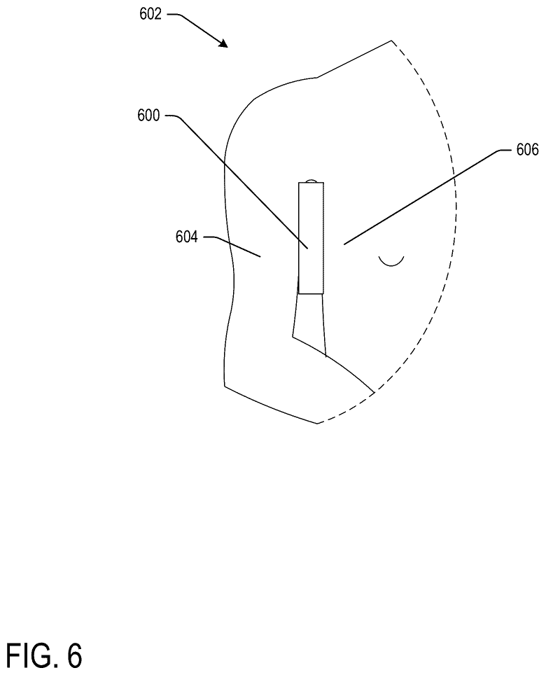

Referring to FIG. 6, an alternative example is illustrated in which a separator 600 is used in the underarm area to separate the upper arm 604 from the chest 606 of a patient 602. In this implementation. the separator 600 may be positioned to create and maintain space between the upper arm 604 and the chest 606 during medical procedures. The separator 600 may have a shape and size adapted to fit comfortably in the axillary region while providing effective separation of the anatomical surfaces.

In some examples, the separator 600 may include angled contact surfaces similar to those described in previous examples, allowing it to conform to the contours of the upper arm 604 and chest 606 while maintaining the desired spacing. The use of the separator 600 in this area may help reduce skin-to-skin contact and potentially decrease radiation exposure to sensitive tissues in the axillary region during treatment procedures.

The separator 600 may be constructed from materials similar to those described for other separator examples, such as air equivalent or radiotransparent materials, to minimize interference with treatment or treatment modalities. In some implementations, the separator 600 may be designed with specific curvatures or contours to better fit the anatomical structures of the underarm area.

The versatility of the separator may extend to various other treatment scenarios. In some implementations, the separator may be used to create space between anatomical regions in other parts of the body. For example, during upper thigh irradiation, the separator may be positioned to separate the genitals from the treatment area. This application may help reduce radiation exposure to sensitive tissues in the groin region during treatment of nearby areas.

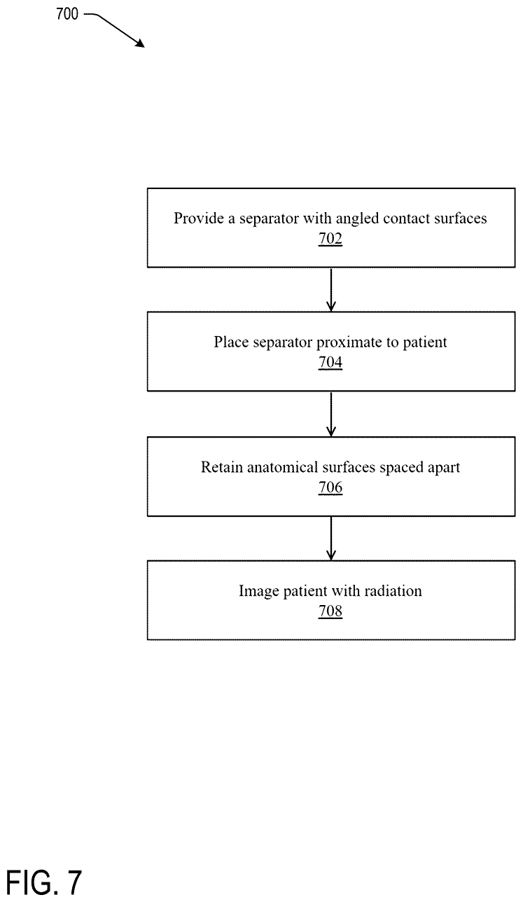

Referring to FIG. 7, a method 700 for medical treatment using a separator is illustrated. The method 700 may include an operation 702, which involves providing a separator. In some examples, the separator may include a first contact surface and a second contact surface that are angled relative to each other. The specific angle between the contact surfaces may be selected based on the desired degree of separation and the particular anatomical region where the separator may be used. Without limit, the separator may be any of the separators 110, 200, 300, 400, 500, 600 described herein.

The method 700 may continue with an operation 704, which involves placing the separator proximate a patient. In some cases, the separator may be positioned such that the first contact surface contacts a first anatomical surface of the patient and the second contact surface contacts a second anatomical surface of the patient. The placement of the separator may vary depending on the specific procedure and anatomical region of interest. For instance, the separator may be placed in the inframammary fold region during breast treatment, or in other anatomical regions where tissue separation may be desired for improved treatment, reduced radiation exposure, and/or the like.

An operation 706 of the method 700 may involve retaining the first anatomical surface spaced from the second anatomical surface. The angled configuration of the separator's contact surfaces may allow for creating and maintaining space between different body regions that may otherwise be in close contact. This separation may be beneficial in various medical treatment scenarios. For example, during treatment of the breast, the separator may be positioned to create space between the breast tissue and the chest wall.

In some examples, the separator may be retained in position through various methods. The weight of the anatomical structure it is supporting may be sufficient to hold the separator in place. For example, when used in breast treatment or treatment, the weight of the breast tissue may keep the separator positioned between the breast and chest wall.

In other cases, the separator may be secured using an adhesive. The adhesive may be applied to one or both contact surfaces of the separator to ensure it remains in the desired position throughout the procedure. The choice of adhesive may depend on factors such as skin sensitivity, duration of use, and ease of removal. In some implementations, a medical-grade adhesive may be used to minimize the risk of skin irritation.

The separator may also be designed with features that aid in retention. For instance, the lip 410 and/or contours of the surfaces 402, 406 mentioned in the description of separator 400 may help secure the device in place, e.g., by creating opposing forces when breast tissue is placed over the lip 410. In other examples of this disclosure, the separator may include textured surfaces or small protrusions that increase friction against the skin, helping to prevent slippage during use.

In certain implementations, the separator may be integrated with or attached to other positioning aids or immobilization devices used during medical procedures. This integration may provide additional stability and ensure consistent placement of the separator across multiple treatment sessions.

The shape and curvature of the separator may also contribute to its retention. The contoured design that conforms to anatomical structures may naturally help keep the separator in place. In some cases, the wedge-like profile may allow the separator to be gently wedged between anatomical surfaces, utilizing the pressure of the surrounding tissues to maintain its position.

The final step of the method, step 708, may involve treating the patient by applying radiation proximate the first anatomical surface. In some examples, the treatment may be performed using various modalities such as radiography, computed tomography (CT), or other radiation-based techniques. The separator may serve to reduce the dose of radiation at the first anatomical surface and/or the second anatomical surface. This reduction in radiation dose may be achieved by creating physical separation between the anatomical surfaces, potentially reducing scatter radiation or direct exposure to certain areas.

In some implementations, the method may include additional steps or variations. For example, the method may involve selecting a separator with specific dimensions or angular relationships between surfaces based on the patient's anatomy or the particular treatment requirements. The method may also include steps for adjusting the position of the separator to optimize spacing and image quality.

In some examples, the method may include steps for ensuring consistent positioning of the separator across multiple treatment sessions. This may involve marking the separator or using anatomical landmarks to guide placement. Such consistency may be particularly important in scenarios where multiple sessions are required, such as during a course of radiotherapy treatments.

In some examples, the separator may provide additional benefits in radiotherapy applications. The separator may help reduce the dose of scatter radiation reaching various anatomical structures during treatment. For example, when used in breast radiotherapy, the separator may reduce the dose of scatter radiation reaching the breast and inframammary fold from the chest. Similarly, the separator may reduce the dose of scatter radiation reaching the chest and inframammary fold from the breast. The use of the separator may also contribute to reducing radiation dose to the skin of the breast. By creating space between the breast tissue and the chest wall, the separator may allow for more of the breast surface to be incident to the radiation beam. This configuration may take advantage of the skin sparing properties offered by incident megavoltage (MV) photon beams, potentially leading to reduced skin dose in the treated area.

In some cases, the separator may enable a reduction in radiation dose to critical organs near the treatment area. For instance, when used in breast radiotherapy, the separator may allow the incident radiation beam aperture to be placed further away from anatomical structures such as the lungs, heart, and ribs. This increased distance may contribute to reducing the dose received by these organs during treatment.

The separator may be adaptable to different treatment modalities and radiation types used in radiotherapy. In some examples, the benefits of reduced scatter radiation and improved tissue sparing may apply to various forms of radiation used in cancer treatment, including but not limited to photon beams, electron beams, and particle therapy.

The ability of the separator to create and maintain space between anatomical surfaces may also contribute to improved dose distribution in the target area. By reducing tissue overlap and creating more uniform surfaces for radiation delivery, the separator may help achieve more precise and consistent dose delivery to the intended treatment volume.

In some cases, the use of the separator in radiotherapy may contribute to improved patient outcomes by potentially reducing treatment-related side effects. The reduction in radiation dose to non-target tissues and organs at risk may help minimize acute and long-term complications associated with radiotherapy treatments.

A number of implementations have been described. Nevertheless, it will be understood that various modifications may be made without departing from the spirit and scope of the disclosure. Accordingly, other implementations are within the scope of the following claims.

What is claimed is:

1. A separator for medical treatment, comprising:
a first contact surface configured for contacting a first anatomical surface of a patient to be imaged; and
a second contact surface configured for contacting a second anatomical surface of the patient to be imaged to space the first anatomical surface from the second anatomical surface,
wherein the first contact surface is angled relative to the second contact surface.

2. The separator of claim 1, wherein:
the first contact surface extends from a first arcuate surface to a second arcuate surface; and
the second contact surface extends from the first arcuate surface to the second arcuate surface.

3. The separator of claim 2, wherein:
the first arcuate surface is substantially parallel to the second arcuate surface.

4. The separator of claim 2, wherein:
at least one of the first contact surface or the second contact surface extends along an arc between a first end wall and a second end wall.

5. The separator of claim 4, wherein the arc is between about 90-degrees and about 190-degrees.

6. The separator of claim 1, wherein the first contact surface is angled relative to the second contact surface by an angle of from about 2-degrees to about 30-degrees.

7. The separator of claim 1, wherein the separator comprises at least one of an air equivalent material or a radiotransparent material.

8. A method of treating a patient, comprising:
providing a separator comprising a first contact surface and a second contact surface angled relative to the first contact surface;
placing the separator proximate a patient such that the first contact surface contacts a first anatomical surface of the patient and the second contact surface contacts a second anatomical surface of the patient, to retain the first anatomical surface spaced from the second anatomical surface; and
treating the patient by applying radiation proximate the first anatomical surface, wherein the separator reduces a dose of the radiation at the first anatomical surface and the second anatomical surface.

9. The method of claim 8, wherein the first contact surface extends from a first arcuate surface to a second arcuate surface, and the second contact surface extends from the first arcuate surface to the second arcuate surface.

10. The method of claim 9, wherein at least one of the first contact surface or the second contact surface extends along an arc between a first end wall and a second end wall.

11. The method of claim 10, wherein the arc is between about 90-degrees and about 190-degrees.

12. The method of claim 8, wherein the first contact surface is angled relative to the second contact surface by an angle of from about 2-degrees to about 30-degrees.

13. The method of claim 8, wherein the separator comprises at least one of an air equivalent material or a radiotransparent material.

14. The method of claim 13, wherein the at least one of the air equivalent material or the radiotransparent material comprises at least one of foam, polyurethane foam, or a polymeric material.

15. A system for medical treatment, comprising:
a medical apparatus configured to emit radiation;
a patient support; and
a separator for use with a patient positioned on the patient support, the separator comprising:
a first contact surface configured to contact a first anatomical surface of a patient; and
a second contact surface angled relative to the first contact surface and configured to contact a second anatomical surface of the patient,
wherein the separator is configured to maintain spacing between the first anatomical surface and the second anatomical surface during treatment.

16. The system of claim 15, wherein the first contact surface extends from a first arcuate surface to a second arcuate surface, and the second contact surface extends from the first arcuate surface to the second arcuate surface.

17. The system of claim 16, wherein at least one of the first contact surface or the second contact surface extends along an arc between a first end wall and a second end wall.

18. The system of claim 17, wherein the arc is between about 90-degrees and about 190-degrees.

19. The system of claim 15, wherein the first contact surface is angled relative to the second contact surface by an angle of from about 2-degrees to about 30-degrees.

20. The system of claim 19, wherein the separator comprises at least one of an air equivalent material or a radiotransparent material.
